(19) United States
(12) Patent Application Publication
(10) Pub. No.: US 2009/0082762 A1
(43) Pub. Date: Mar. 26, 2009

(54) RADIO FREQUENCY ENERGY TRANSMISSION DEVICE FOR THE ABLATION OF BIOLOGICAL TISSUES

(76) Inventors: Theodore C. Ormsby, Escondido, CA (US); George L. Leung, San Diego, CA (US); Ming Fan Law, San Diego, CA (US)

Correspondence Address: PROCOPIO, CORY, HARGREAVES & SAVITCH LLP, 530 B STREET, SUITE 2100, SAN DIEGO, CA, 92101, US

(21) Appl. No.: 11/858,736

(22) Filed: Sep. 20, 2007

(51) Int. Cl. A61B 18/18

(52) U.S. Cl. 606/33

(57) ABSTRACT

A radio frequency energy transmission device comprises a hollow coaxial electrically conductive cable adapted for conduction of radio frequency (RF) energy, particularly microwave energy, for the ablation of biological tissue. The hollow cable has a proximal end and a distal end and comprises coaxial inner and outer conductors extending substantially the entire length of the cable from the proximal end to a distal end portion of the cable. The inner conductor comprises an elongated electrically conductive tubular member having a hollow, axially extending lumen, and the outer conductor comprises an elongated electrically conductive tubular member disposed in a substantially coaxial relationship over at least a portion of the inner conductor. Dielectricity to impede conduction between the inner and outer conductors is introduced with a vacuum or dielectric medium disposed between the inner and outer conductors. An ablating member, which delivers radio frequency energy, particularly microwave energy, to body tissue is disposed at a distal end portion of the cable. The ablating member can be a helical coil, a monopole of a microstrip circuit.

### BACKGROUND

1. Field of the Invention

The present invention generally relates to medical devices, which are used for the irradiation of biological tissues, such as devices for the ablation of biological tissues, and more particularly to a radio frequency energy transmission device for such devices.

2. Related Art

Therapeutic issue ablation systems apply energy to a biological ablation tissue site via different energy exchange means, such as heat conduction and irradiation. These systems may employ various energy modes, such as radiofrequency, ultrasound, laser, cryogenic, and the like. Within the radio frequency (RF) range, certain microwave ablation systems are used to destroy or ablate biological tissues. In one application, a microwave ablation system is used to ablate cardiac tissues that cause irregular heartbeats or arrhythmia, avoiding the need for more risky and invasive open heart surgery. In such an application, an ablation member such as an RF antenna is incorporated as part of a catheter. The catheter is passed through the vein for access to the atrium. Within the atrium, the RF antenna is positioned at the desired location where ablation is applied.

Microwave ablation systems can also be used in treatment of other biological sites such as arteries, organs and body vessels. As an example, a microwave ablation system is used to ablate tumors in the lungs, liver, kidney or other areas of the body.

These surgical and therapeutic applications require an efficient system for the transmission of radio frequency energy to the ablating member for the delivery of energy to the target tissue site.

### SUMMARY

The present invention provides an innovative radio frequency energy transmission device for the ablation of biological tissues in body areas such as the heart, liver, and the like. The embodiments described herein provide a new conductive hollow coaxial cable device with a central lumen for use in a radio frequency based tissue ablation system.

In one embodiment, a hollow conductive coaxial cable is provided. It comprises a first inner elongated electrically conductive tubular member having an axially extending lumen or passageway. A second elongated electrically conductive member is disposed in a substantially coaxial relationship over at least a portion of the first electrically conductive tubular member. Between the inner and outer conductive members, a dielectric medium is provided. At the distal end portion of the cable, an ablating member is mounted for the delivery of radio frequency energy including microwaves to the target body tissue.

In one embodiment, the ablating member comprises a radio frequency transmitter or antenna, which may be a helical coil, or a monopole, having one end connected to the inner conductive member and a second end connected to the outer conductive member. A radio frequency signal generator is connected to the proximal end of the cable to generate a train of RF pulses along the cable to the RF antenna, along with a controller or control unit for adjusting the RF signal according to predetermined parameters. In one embodiment, the radio frequency may be a microwave frequency from approximately 300 MHz and up.

In one embodiment, a dielectric medium is selectively disposed between the inner and outer conductors. The dielectric medium may comprise a solid or a fluid material, or a combination of both and may assume alternative structure features.

An ablating member for delivery of radio frequency energy to the target biological tissue site, particularly microwave energy, is mounted at the distal end portion of the cable.

Other features and advantages of the present invention will become more readily apparent to those of ordinary skill in the art after reviewing the following detailed description and accompanying drawings.

### BRIEF DESCRIPTION OF THE DRAWINGS

FIG. 1 is a schematic block diagram, partially broken away, illustrating one embodiment of radio frequency energy transmission device for the ablation of biological tissues;

FIG. 2 is a longitudinal cross-sectional view of a first embodiment of a hollow conductive coaxial cable for the device of FIG. 1;

FIG. 3 is a cross-section taken on the lines 3-3 of FIG. 2;

FIG. 4 is a cross-section taken on the lines 4-4 of FIG. 2;

FIG. 5-1 is a partial isometric sectional view of a modified hollow conductive coaxial cable in which a dielectric layer is disposed between the inner and the outer electrical conductors of the cable;

FIG. 5-2 is a selected cross-sectional view of the modified hollow conductive coaxial cable shown in FIG. 5-1;

FIG. 5-3 is a cross-sectional view of another embodiment of the hollow conductive coaxial cable with two separate dielectric layers disposed between the inner and outer electrical conductors;

FIG. 5-4 is a cross-sectional view of a further alternative embodiment of the hollow conductive coaxial cable illustrating a plurality of dielectric layers disposed between the inner and outer electrical conductors;

FIG. 6-1 is a cross-sectional view of another variation of embodiment of the hollow conductive coaxial cable with an alternative dielectric layer disposed between the inner and outer electrical conductors;

FIG. 6-2 is a cross-sectional view of the dielectric material for use in the embodiment illustrated in FIG. 6-1;

FIG. 6-3 is a partial isometric sectional view of the dielectric material for use in the illustrated in FIGS. 6-1 and 6-2;

FIG. 7-1 is a cross-sectional view of another embodiment of the dielectric material for placement between the inner and outer electrical conductors of the present invention;

FIG. 7-2 is a partial isometric sectional view of the dielectric material for use in the embodiment illustrated in FIG. 7-1;

FIG. 7-3 is a cross-sectional view of a further alternative embodiment of the dielectric material for placement between the inner and outer electrical conductors of the present invention; and

FIG. 7-4 is a partial isometric sectional view of the dielectric material for use in the embodiment illustrated in FIG. 7-3.

### DETAILED DESCRIPTION

The present invention provides an innovative radio frequency energy transmission device, which incorporates a hollow coaxial cable for conducting radio frequency (RF) energy, particularly microwave energy, for the ablation of biological tissues. The hollow cable has a proximal end and a distal end and comprises coaxial inner and outer conductors. The inner conductor has an elongated electrically conductive tubular member with a hollow, axially extending lumen. The outer conductor has an elongated electrically conductive tubular member, which is arranged in a substantially coaxial relationship over the inner conductor. A dielectric medium is selectively disposed between the inner and outer conductors. An ablating member which delivers radio frequency energy, particularly microwave energy, at the distal end portion of the cable. The hollow conductive coaxial cable is adapted to connect with an RF signal generator at a proximal end and delivers the RF energy, particularly microwave energy to an ablation member mounted at a distal end portion.

FIGS. 1-3 illustrate a radio frequency energy transmission (RF) energy ablation system 100, which comprises an elongated coaxial cable device 20 adapted for placement adjacent to or within a biological tissue site and/or a body vessel of a patient and an ablation device 60, such as an RF antenna, for delivering electromagnetic energy to the treatment site, as described in more detail below.

The coaxial cable device 20 has a flexible, elongated tubular body 32 having a proximal end portion 25 and a distal end portion 30. Located at the proximal end portion of the coaxial cable device is a handle unit 40, which contains steering and positioning controls (not illustrated) for the coaxial cable device. An RF signal generator and system control unit or system 35 is connected to the proximal end of the coaxial cable device by cable 45, and is electrically coupled to the ablation device 60 through the coaxial cable, as described in more detail below. The RF signal generator and control unit for controlling the RF signal delivered to the ablation device may be as described in the pending application Ser. No. 11/479,259 filed on Jun 30, 2006, the contents of which are incorporated herein by reference.

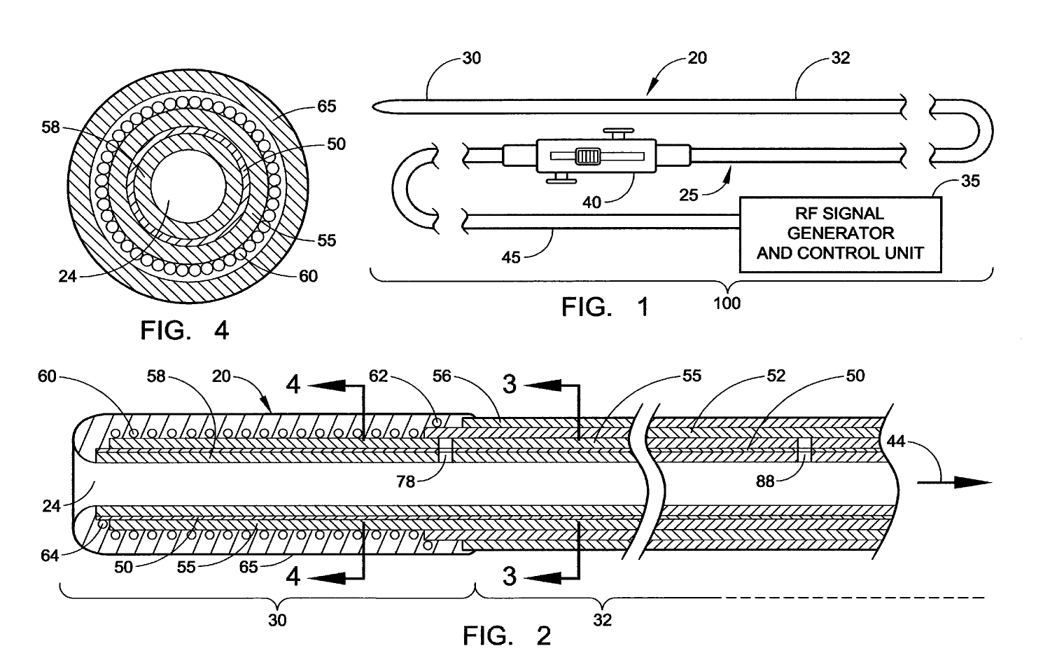

The structure of one embodiment of the coaxial cable device 20 is illustrated in more detail in FIGS. 2 to 4. The length and diameters of coaxial cable device 20 are adapted as required to suit the particular medical procedure, as is known in the medical art. Coaxial device 20 is generally tubular and has a multi-layer construction with a central bore or lumen 24 extending along its length. The distal end 30 of the lumen 24 may be close as illustrated in FIGS. 2 or it may be open in other embodiments, for example as described and shown in U.S. Pat. No. 6,663,625, the contents of which are incorporated herein by reference.

The coaxial cable device 20 comprises a first or inner electrically conductive tubular member or conductor 50 having a proximal end portion and a distal end portion. Inner conductor 50 is constructed of an elongated electrically conductive tubular member having a hollow lumen 24. An outer conductor 52, also made of an elongated electrically conductive tubular member, is arranged in a substantially coaxial relationship over at least a portion of length of the inner conductor 50. This arrangement defines a space 54 between the walls of the inner conductor 50 and the outer conductor 52.

An ablation device 60 is located at the distal end portion 30 of the coaxial cable device 20 and is electrically coupled to both the outer coaxial conductor 52 at contact point 62 and to the inner conductor 50 at contact point 64. In turn, the inner conductor and the second or outer conductor are electrically coupled to the RF energy source in unit 35. In the illustrated embodiment, the ablation device 60 comprises a helical coil wound around the outer circumferential surface of the coaxial cable device and extending from the end portion of the outer conductor 52 up to the distal end portion or tip of the device 20. The helical coil 60 is coated with an outer coating layer 65 of dielectric material such as a polymeric dielectric encapsulant which protects the structural integrity of the coil and also shields it from the surrounding biological environment. In alternative embodiments, other forms of ablation devices or radio frequency antennas may be used in place of the helical coil antenna 60, such as a monopole bead antenna or a pair of spaced electrically conductive microstrips disposed at the distal end portion of the coaxial cable device, as described in U.S. Pat. No. 6,663,625 referenced above, the contents of which are incorporated herein by reference. The RF antenna 60 includes an electrically conductive material or wire strip that is wound in a helical fashion to form a helical coil. The appropriate diameter, pitch and length of the coil winding, and the selection of the conductive material or wire strip are a matter of choice, which can vary according to the particular procedure requirements as known in the art. Thus these design elements and considerations are not detailed here.

As shown in FIGS. 1-3, a dielectric medium 53 is provided in space 54 to impede electrical conduction between the inner conductor 50 and outer conductor 52. The dielectric medium is formed from a solid or a fluid or a combination of solid and fluid. Selectively, the dielectric is formed of a dielectric layer 55, which substantially fills the space 54 between the inner conductor 50 and outer conduction 52, with unfilled space in vacuum or filled with an alternative dielectric solid or fluid material. A dielectric fluid medium such as air may be dispensed in lieu of the solid dielectric layer 55. Vacuum, which also exhibits dielectric property, may be introduced by the evacuation of air and sealing the space 54 between the distal and proximal end portions of the cable during manufacture. Alternately, the vacuum can be effected by means of a vacuum source configured in fluid communication with space 54, as discussed in more detail below.

An outer jacket or casing 56 encases the outer conductor 52 along the length of the coaxial cable device up to the distal end portion 30. The outer casing 56 is generally constructed of a polymer material that is bio-compatible within the body vessel environment. Examples of such materials include thermoplastic elastomer material such as Pebax® available from Autochem Germany, polyethylene, polyurethane, polyester, polyimide, polyamide, and the like, with varying degrees of radiopacity, hardness, and elasticity.

The tubular body of the coaxial cable device 20 may be formed with a plurality of segments using one or more of the aforementioned materials or equivalents, such that the device 20 is progressively more flexible towards its distal end. The segments may be joined together by thermal bonding, butt joints, or adhesive bonding. Braiding reinforcement may be provided to the surface of the tubular body to attain a desirable level of stiffness and torsional strength for the device to advance and negotiate through the body vessel of the patient, while still allowing the distal end portion to be bent when needed. The distal end portion 30 may be of a softer polymer compound than the remainder of the body, with little or no braiding or reinforcement, to provide the desired flexibility for distal deflection and shaping of the apparatus.

In one embodiment, inner conductor 50 may be made of a flexible braided wire construction or thin film electrically conductive material. An inner liner or sleeve 58 of flexible dielectric material may be provided inside conductor 50 to surround the hollow central bore or lumen 24. The outer conductor 52 may be of a braided wire construction or may be a thin film electrically conductive material or the like. The sleeve 58, the inner conductor 50, and the dielectric layer 55 extend from handle unit 40 through the distal end portion of the coaxial cable device, while the outer conductor 52 and outer casing 56 extend from the handle unit 40 and terminate short of the distal end of the device, with the outer conductor projecting a short distance beyond the distal end of the outer casing, as seen in FIG. 2.

The RF antenna 60 is adapted to receive and radiate electromagnetic energy from a source of radio frequency energy (not shown) in unit 35. An example of suitable spectrum of radio frequency is that of the microwave frequency ranging from approximately 300 MHz and up. The RF antenna 60 imparts substantially uniformly distributed electromagnetic field energy transmitted by the helical coil. The power of the electromagnetic field transmitted is substantially normal to the longitudinal axis of the RF antenna, and a uniform energy field is produced circularly about and bounded by the antenna. The energy delivered for the ablation is substantially uniformly distributed along the antenna, which is independent of the contact between the antenna and the tissue to be ablated.

FIGS. 5-1 and 5-2 show another embodiment of the present invention, which incorporates an alternative dielectric medium configuration. Like reference numerals in FIGS. 5-1 and 5-2 are used for like parts in other figures as appropriate. In this embodiment, the dielectric medium 53 is constructed of a dielectric layer 70, which is disposed in the space 54 between the inner and outer conductors to wrapping around the inner conductor 50. A gap 76 is provided between the longitudinal peripheral edges 72 and 74 of the dielectric layer 70. Gap 76 extends along at least a portion of the length of the coaxial cable and is generally oriented in parallel with the axis of the cable, though other directional alignment can be provided. Additionally, the peripheral edges 72 and 74 of the dielectric layer 70 may be joined in selected locations to define a plurality of voids along the seam of the peripheral edges in the space 54 between the inner conductor 50 and outer conductor 52.

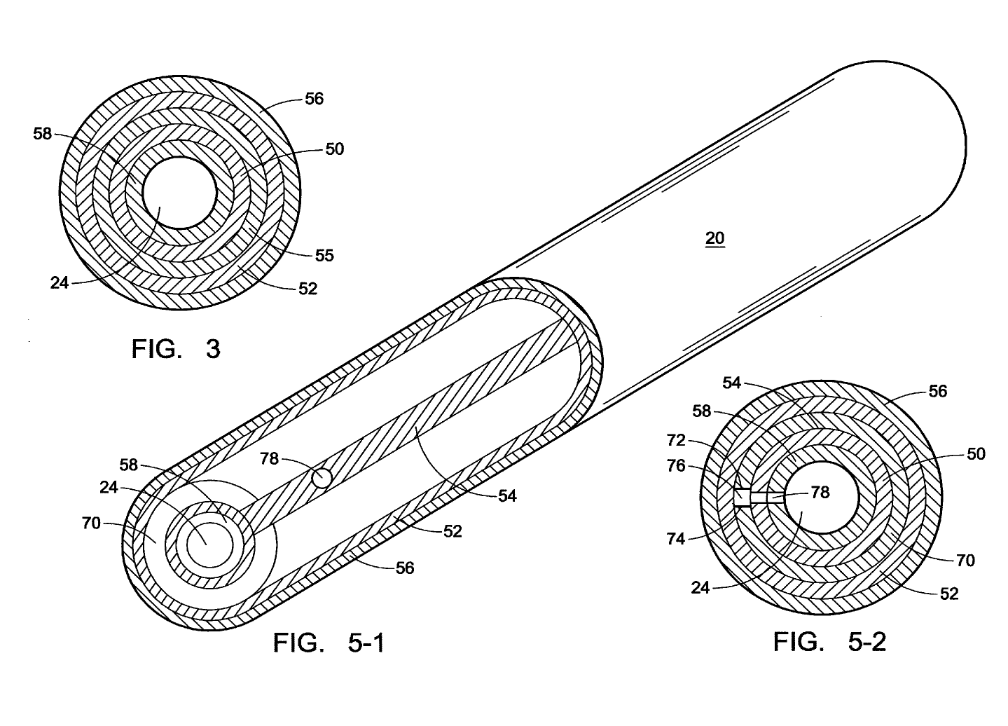

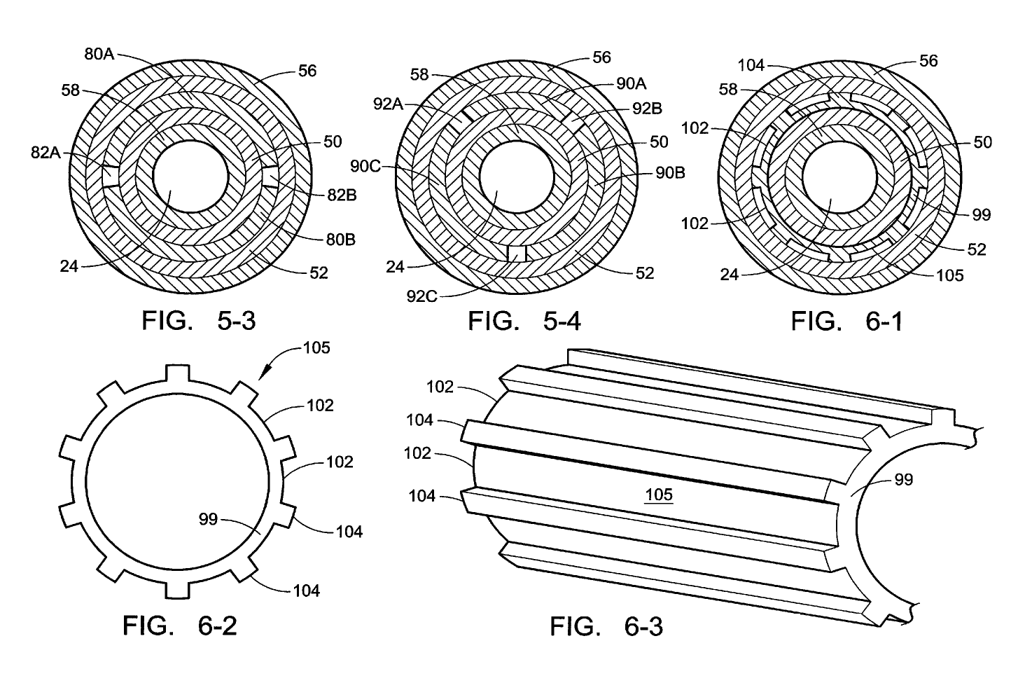

FIGS. 5-3 and 5-4 show the cross-sectional views of additional embodiments of the hollow conductive coaxial cable wherein two or more separate dielectric layers are disposed between the inner and outer electrical conductors. FIG. 5-3 illustrates a configuration where two pieces of dielectric layers 80A, 80B are provided in space 54. The two dielectric layers are separated by gaps 82A and 82B. Similar to the embodiment shown in FIGS. 5-1 and 5-2, gaps 82A and 82B each extend along at least a portion of the length of the coaxial cable in a generally parallel direction with the axis of the inner and outer conductors, though other directional alignment can be provided. Gaps 80A and 80B thus provide elongated channels between the dielectric layers along the length of the coaxial cable in the space 54 between the inner and outer conductors.

In FIG. 5-4, three pieces of dielectric layers 90A, 90B, 90C are provided in space 54. The dielectric layers are separated by gaps 92A, 92B and 93C. Similar to the embodiments shown in FIGS. 5-1-5-4, the orientation of the gaps 92A, 92B and 92C extends in a generally parallel direction with the axis of the inner and outer conductors, though other directional alignment can be provided.

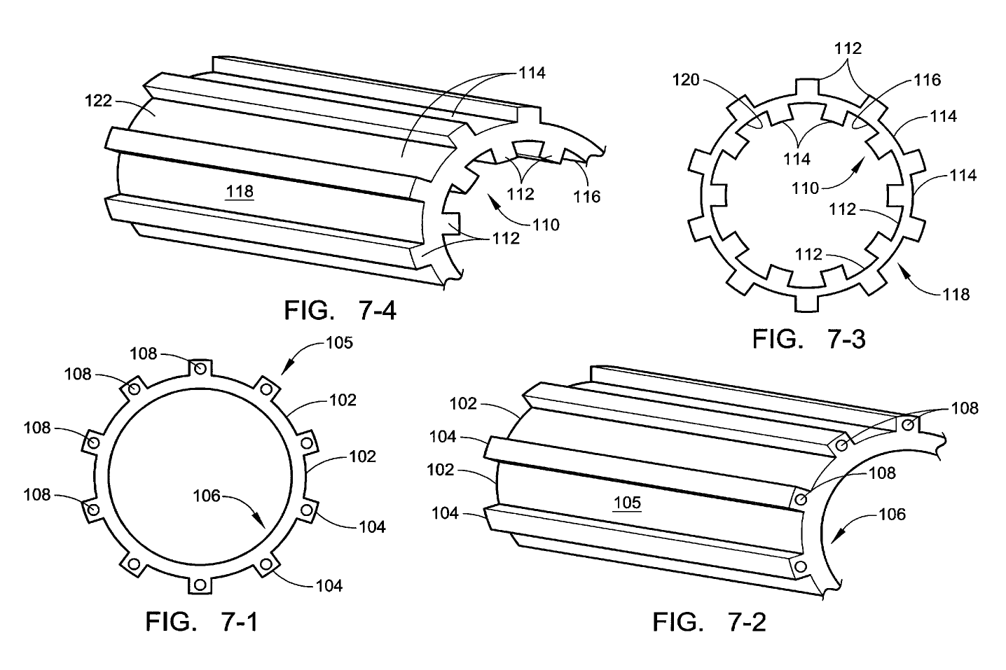

FIG. 6-1 shows a further embodiment of the present invention, which incorporates an alternative dielectric material configuration. A dielectric layer 99 is provided with one or more or surface recesses 102 and is disposed between the inner conductor 50 and the outer conductor 52. As exemplified by the embodiment illustrated in the cross-sectional and partial isometric sectional view of FIGS. 6-2 and 6-3, the recesses 102 are formed between the elongated spines or upraised ridges 104, which extend in a substantially parallel relationship with the axis of the inner and outer conductors. This embodiment defines at least one channel extending between the distal and the proximal end portions of the coaxial cable. As shown in the embodiment illustrated in FIGS. 6-1, 6-2 and 6-3, spines 104 are arranged in an equal-angular relationship about the axis of the coaxial cable. Spines 104 can be formed as part of the dielectric layer 99. Alternatively, they can be formed as separate elongated strips and affixed on the surface 105 of the dielectric layer 99. Further, spines 104 can assume various cross-sectional profiles, which are not limited to those as shown in FIGS. 6-1, 6-2, 6-3, 7-1, 7-2, 7-3 and 7-4.

Optionally, the recesses 102 can be formed and oriented to extend in a spinal fashion relative to the axis of the inner and outer conductors, thus defining one or more spinal channels or passageways in space 54 (not shown) between the inner conductor 50 and the outer conductor 52. As a further alternative design, the lineal recesses can be formed in an intersecting crisscross fashion on either one side or both sides (not shown) of the dielectric layer 100 disposed between the inner conductor 50 and outer conductor 52. Further, in lieu of indentation, lineal or otherwise, formed on the surface of dielectric material, the recesses may be in the form of perforations or voids (not shown).

FIGS. 7-1 and 7-2 illustrate a dielectric layer configuration 106 in which at least one internal passageway 108 is selectively formed in the elongated ridges 104 and extending along the length of the ridges 104 to follow the length of the coaxial cable. This alternative dielectric configuration provides at least one open channel 102 between the elongated spines and at least one internal passageway 108 extending between the distal and the proximal end portions of the coaxial cable.

FIGS. 7-3 and 7-4 illustrate another variation of the embodiment of the present invention where an alternative dielectric layer 110 configured in the space 54 between the inner conductor 50 and the outer conductor 52 is provided with one or more or surface recesses 114 on both surfaces of the dielectric layer 110. Similar to the embodiments described above, the recesses are formed between elongated ridges 112 which extend on the inner surface 116 and outer surface 118 of the dielectric layer 110 along the longitudinal direction of the coaxial cable. This embodiment provides elongated inner channels 120 and outer channels 122 between the distal portion and the proximal portion of the coaxial cable from the distal portion to the proximal portion of the coaxial cable.

In the embodiments presented herein and in the references incorporated hereto, the inner conductor 50 and outer conductor 52 are configured in a substantially coaxial relationship in which the walls between the conductors define a space 54 extending in the length of the coaxial cable. As discussed above, the space 54 is configured to interpose dielectricity, which impedes electrical conduction between the inner and outer conductors, which may be effected with the introduction of a vacuum or a dielectric medium. With respect to a dielectric medium, it can comprise a solid dielectric layer which is disposed between the space between the inner conductor 50 and the outer conductor 52. Alternatively, in lieu of the sold dielectric layer, a dielectric fluid medium can be used. Further, where the gaps and recesses are provided as in the various embodiments as exemplified above, one or more solid dielectric layer(s) and a fluid (such as air) can be placed in space 54.

Optionally one or more access openings can be formed on the distal portion and/or proximal portion of the coaxial cable to provide communication between space 54 and hollow lumen 24. As illustrated in FIGS. 5-1 and 5-2, the access opening 78 at the distal portion and access opening 88 at the proximal portion are selectively formed on the inner conductor 50 and inner liner 58. Such a feature provides an enhanced versatility to the ablation device to enable access to the space between the inner cable and the outer cable. It also provides an additional means to facilitate the introduction of vacuum or dielectric fluids, placement of devices and instruments and dispensing of medication, such as drugs, saline and sterile water to the patient in support of the ablation operation.

The outer dimensions of the body of the coaxial cable device in each of the above embodiments may be adapted as required to suit the particular medical procedure, as is well known in the medical art. In one embodiment, the device is used to ablate cardiac tissue. However, the device may be used to ablate other types of body tissue in different organs, both internal and external to the body. The tubular body of the coaxial cable device may be generally constructed of a polymer material which is bio-compatible with the body vessel environment.

In each of the above embodiments, the ablation device or RF antenna is adapted to receive and radiate electromagnetic energy in order to treat a selected biological tissue site by changing a property of the biological tissue at the site. An example of a suitable spectrum of radio frequency energy for use in tissue ablation is that of the microwave frequency range above 300 MHz. The RF antenna is capable of applying substantially uniformly distributed electromagnetic field energy along the RF antenna in a direction substantially normal to the longitudinal axis of antenna 60. The elongated, flexible coaxial cable device connected to an RF source and control unit at its proximal end extends to a distal end portion at which the RF antenna is mounted. The coaxial cable device in each of the foregoing embodiments has coaxial inner and outer conductors extending from its proximal end and separated by a dielectric medium, and a central lumen or bore inside the inner conductor extends the length of the coaxial cable device and can be used to accommodate conductor wires which are connected to ECG electrodes, temperature sensors, or the like, as well as a suitable shaping or steering mechanism for controlling the shape or deflection of the distal end portion of the coaxial cable device in which the RF antenna is located.

The above description of the disclosed embodiments is provided to enable any person skilled in the art to make or use the invention. Various modifications to these embodiments will be readily apparent to those skilled in the art, and the generic principles described herein can be applied to other embodiments without departing from the spirit or scope of the invention. Thus, it is to be understood that the description and drawings presented herein represent a presently preferred embodiment of the invention and are, therefore, representative of the subject matter which is broadly contemplated by the present invention. It is further understood that the scope of the present invention fully encompasses other embodiments that may become obvious to those skilled in the art and that the scope of the present invention is accordingly limited by nothing other than the appended claims.

1. A radio frequency energy transmission device for the ablation of biological tissues, comprising:
a hollow coaxial electrical cable having a proximal end portion and a distal end portion and comprising (i) an inner conductor comprising an elongated electrically conductive tubular member having a hollow lumen; and (ii) an outer conductor comprising an elongated electrically conductive tubular member disposed in a substantially coaxial relationship over at least a portion of the inner conductor and defining a space between the walls of the inner conductor and the outer conductor wherein a dielectric is interposed;
and
an ablating member electrically coupled to the hollow coaxial cable, which transmits radio frequency energy to the biological tissue.

2. The device of claim 1 wherein the radio frequency comprises that of the microwave frequency from approximately 300 mHz and up.

3. The device of claim 1, wherein the RF antenna comprises a helical coil wound around the distal end portion of the cable.

4. The device of claim 1, wherein the distal end of the hollow lumen is open.

5. The device of claim 1, wherein the distal end of the hollow lumen is closed.

6. The device of claim 1, wherein at least one of the conductive tubular members is formed of an electrically conductive wire mesh.

7. The device of claim 1, wherein at least one of the conductive tubular members is formed of an electrically conductive braided material.

8. The device of claim 1, wherein at least one of the conductive tubular members is formed of an electrically conductive thin-film material.

9. The device of claim 1, wherein the space between the inner and outer conductors defines a vacuum.

10. The device of claim 1, wherein the space between the inner and the outer conductors is in fluidic communication with a source of vacuum.

11. The device of claim 1, wherein the space between the inner and the outer conductors is in fluid communication with the hollow lumen.

12. The device of claim 1, further comprising a dielectric medium disposed between the inner conductor and the outer conductor.

13. The device of claim 12, wherein the dielectric medium is formed from a solid or a fluid or a combination of solid and fluid.

14. The device of claim 13, wherein the dielectric medium comprises a dielectric layer selectively disposed between the inner and outer conductors.

15. The device of claim 14, wherein the solid dielectric layer substantially fills the space between the walls of the inner conductor and outer conductor.

16. The device of claim 14 wherein the dielectric medium further comprises recesses selectively formed on at least one of the surfaces of the dielectric layer.

17. The device of claim 16, wherein the recesses are formed to extend in a substantially parallel relationship with the axis of the cable.

18. The device of claim 16, wherein the recesses are formed on both sides of the dielectric layer.

19. The device of claim 16, wherein the recesses are formed in a crisscross fashion.

20. The device of claim 16, wherein at least one of the recesses formed on the dielectric medium is in fluid communication with the hollow lumen.

21. The device of claim 12, wherein the dielectric medium comprises one or more elongated ridge members disposed in an equal-angular relationship about the axis of the cable and aligned substantially parallel to the axis of the coaxial cable.

22. The device of claim 21, wherein at least one or more elongated ridge members comprises a passageway extending in parallel to the axis of the coaxial cable.

23. The device of claim 1, wherein the ablation member comprises a monopole bead.

24. The device of claim 1, wherein the ablation member comprises a pair of spaced electrically conductive microstrips.

25. A radio frequency ablation apparatus, comprising:
an elongated hollow coaxial cable having a proximal end and a distal end adapted for the transmission of radio frequency (RF) energy for the ablation of biological tissues, comprising:
a radio frequency (RF) antenna disposed at the distal end portion of the cable which receives input RF energy for ablation of biological tissue;
an electrical connector at the proximal end of the cable which connects the cable to an RF signal generator for the RF antenna;
inner and outer coaxially aligned, circumferentially spaced, electrically conductive tubular members extending through the cable from the proximal end to the RF antenna which connect the RF antenna to the RF signal generator through the electrical connector, the inner tubular member having a hollow, axially extending lumen which extends from the proximal end to the distal end portion of the cable; and
means to interpose a dielectric between the inner and outer electrically conductive tubular members.
